(19) United States
(12) Patent Application Publication
(10) Pub. No.: US 2026/0365608 A1
(43) Pub. Date: Nov. 19, 2026

(54) PROCESS FOR THE PRODUCTION OF AN ALKYLSTYRENE AND OF AN ALKYLPHENYL PROPIONIC ACID

(71) Applicant: BASF SE, Ludwigshafen, DE (US)

(72) Inventors: Michael Puhl, Ludwigshafen (DE); Natalia Trukhan, Ludwigshafen (DE); Tim Massmann, Bishop, TX (US); Katharina Stefanie Ludwina Rueck, Ludwigshafen (DE); Thomas Fenlon, Ludwigshafen (DE)

(21) Appl. No.: 19/137,320

(22) PCT Filed: Dec. 12, 2023
(86) PCT No.: PCT/EP2023/085328

§ 371 Date: Jun. 10, 2025

(30) Foreign Application Priority Data
Dec. 13, 2022 (EP) 22213122.9

(51) Int. Cl. C07C 57/30; B01J 6/00; B01J 29/70; B01J 37/00; B01J 37/04; C07C 4/06; C07C 7/00; C07C 15/04; C07C 15/46; C07C 51/14

(52) U.S. Cl. CPC C07C 57/30; B01J 6/001; B01J 29/7038; B01J 37/0009; B01J 37/009; B01J 37/04; C07C 4/06; C07C 7/00; C07C 15/04; C07C 15/46; C07C 51/14; B01J 2229/10; C07C 2529/04

(57) ABSTRACT

The present invention relates to a process for the production of an alkylstyrene comprising (1) providing a feed containing a 1,1-diarylethane compound, wherein the aryl-group is an al-kylphenyl group; (2) providing a catalyst comprising a zeolite having the MWW-type framework structure; (3) contacting the feed provided in (1) with the catalyst provided in (2) for the catalytic cracking of the 1,1-diarylethane to the alkylstyrene and the alkylbenzene; and (4) separating the alkylstyrene from the reaction product obtained in (3). The present invention further relates to a process for the production of an alkylphenyl propionic acid comprising steps (1) to (4).

### TECHNICAL FIELD

The present invention relates to a HF and AC₃ free process for the production of alkylstyrenes with zeolites as heterogeneous catalysts. Further, the present invention relates to a process for the production of alkylphenyl propionic acids using the obtained alkylstyrene and the regeneration of the zeolite catalyst.

### INTRODUCTION

Alkylstyrenes are an important commodity chemical, which find wide application as co-monomers for synthetic plastics or as intermediates in the organic chemical industry.

Alkylstyrenes can be used as precursor for the production of alkylphenyl propionic acids via catalytic cracking of 1,1-diaryl ethane. A process for the production of a-(p-isobutylphenyl) propionic acid or its alkyl esters is described in U.S. Pat. No. 4,694,100. According to this process, in a first step isobutylbenzene is reacted with acetaldehyde in the presence of sulphuric acid to produce 1,1-bis(p-isobutylphenyl) ethane. The produced 1,1-bis(p-isobutylphenyl) ethane is then catalytically cracked in the presence of a protonic acid catalyst into isobutylbenzene and p-isobutylstyrene. According to this document, p-isobutylstyrene is reacted with carbon monoxide and water or alcohol to produce α-(p-isobutylphenyl) propionic acid or its alkyl esters.

Further, EP 0300498 A describes an optimized first step, wherein hydrogen fluoride is used as catalyst instead of sulphuric acid.

The process in EP 0316014 A, on the other hand, concerns the recycling of 1,1-bis(p-isobutylphenyl)ethylene to 1,1-bis(p-isobutylphenyl) ethane with hydrogen and a hydrogenation catalyst. 1,1-bis(p-isobutylphenyl)ethylene is formed as by product during the catalytic cracking of 1,1-bis(p-isobutylphenyl) ethane as described in U.S. Pat. No. 4,694,100.

EP 0015758 A1 describes a process for producing styrene or nucleus-substituted styrene by the catalytic decomposition of 1,1-diarylethane in gaseous phase in the presence of zeolite as catalyst. Zeolites of different framework types are used: FAU, MOR or CHA.

WO 2017/009458 A1 describes a gas phase thermolysis process for preparing an arylpropene from a 1,1-diarylpropane wherein a thermolysis catalyst is employed which comprises a boron containing zeolitic material with a BEA or MWW framework type.

US 2018/134570 A1 describes the production of a boron-containing zeolitic material having an MWW framework structure.

Despite the advances made with regard to the production of alkylstyrenes and alkylphenyl propionic acids, there remains the need for a highly efficient process for their respective production. In particular, there remains the need for improved catalysts displaying improved activities and selectivity's, as well as longer lifetimes and regeneration stability. In addition thereto, there remains the need for the provision of such highly efficient processes without involving the use of costly compounds, or of compounds which might pose a health hazard or might be harmful for the environment.

### DETAILED DESCRIPTION

Thus it was the object of the present invention to provide an improved process for the production of alkylstyrenes and alkylphenyl propionic acids, and particular with regard to the activity and selectivity thereof, as well as with regard to the lifetime of the catalyst on stream and to the possibility of regenerating the catalyst without a substantial loss in catalytic efficiency. Furthermore, it was the object of the present invention to provide a process which avoids the use of compounds which may pose a problem to the health of employees and/or to the environment. Surprisingly, it was found that the process of the present invention permits an efficient synthesis of p-isobutylstyrene using zeolite catalysts with high product selectivities towards the targeted styrene and the conformer of the 1,1-diarylethane at high conversion rates. Further, the process reduces the amount of p-isobutylethylbenzene as side component, which has comparable properties to the desired product and makes distillated separation difficult.

Yet further, it has quite unexpectedly been found that the catalyst lifetime of zeolite catalysts having a particular type of framework structure and composition is longer compared e.g. to impregnated silica gel catalysts. Furthermore, regeneration of conventional catalysts often leads to a washing out effect, whereas the zeolite catalysts of the inventive process can be recycled through calcination. Furthermore, no hydrogen fluoride or hydrogen gas is required as e.g. described in EP 0300498 A, which reduces the necessary safety requirement of the process of the present invention.

Therefore, the present invention relates to a process for the production of an alkylstyrene comprising
(1) providing a feed containing a 1,1-diarylethane compound, wherein the aryl-group is an alkylphenyl group;
(2) providing a catalyst comprising a zeolite having the MWW-type framework structure;
(3) contacting the feed provided in (1) with the catalyst provided in (2) for the catalytic cracking of the 1,1-diarylethane to the alkylstyrene and the alkylbenzene;
(4) separating the alkylstyrene from the reaction product obtained in (3).

Furthermore and independently thereof, the present invention relates to a process for the production of an alkylphenyl propionic acid comprising
(1) providing a feed containing a 1,1-diarylethane compound, wherein the aryl-group is an alkylphenyl group;
(2) providing a catalyst comprising a zeolite having the MWW-type framework structure;
(3) contacting the feed provided in (1) with the catalyst provided in (2) for the catalytic cracking of the 1,1-diarylethane to the alkylstyrene and the alkylbenzene;
(4) optionally separating the alkylstyrene from the reaction product obtained in (3);
(5) preparing a mixture containing CO, the alkylstyrene-containing reaction product obtained in (3) or the alkylstyrene obtained in (4), and optionally a solvent system;
(6) providing a carbonylation catalyst;
(7) contacting the mixture obtained in (5) with the carbonylation catalyst provided in (6) for converting the alkylstyrene to the alkylphenylpropionic acid.

It is preferred that in (1) the alkyl group of the alkylphenyl group is selected from the group consisting of C1-C10 alkyl, preferably C2 to C6 alkyl, more preferably C3 to C5 alkyl, and more preferably C4 alkyl, wherein more preferably the alkyl group of the alkylphenyl group is selected from the group consisting of ethyl, n-propyl, i-propyl, n-butyl, i-butyl, and 1-methylpropyl, more preferably from the group consisting of i-propyl, n-butyl, i-butyl, and 1-methylpropyl, more preferably from the group consisting of i-propyl, i-butyl, and 1-methylpropyl, wherein more preferably the alkyl group of the alkylphenyl group is i-butyl.

It is preferred that in (1) the alkylphenyl group is selected from the group consisting of o-isobutylphenyl, m-isobutylphenyl, and p-isobutylphenyl, wherein preferably the alkylphenyl group is pisobutylphenyl.

It is preferred that the zeolite in (2) is selected from the group consisting of PSH-3, SSZ-25, ERB-1, MCM-22, ITQ-1, SSZ-25, [Ga—Si—O]-MWW, [B—Si—O]-MWW, [Sn—Si—O]-MWW, [AI-Si—O]-MWW, [Zr—Si—O]-MWW, [V—Si—O]-MWW, [Nb—Si—O]-MWW, [Ta—Si—O]-MWW, [Cr—Si—O]-MWW, [Mo—Si—O]-MWW, [W—Si—O]-MWW, [Mn—Si—O]-MWW, [Fe—Si—O]-MWW, [Co—Si—O]-MWW, [Ni—SiO]-MWW, [Zn—Si—O]-MWW, [Ga—Si—O]-MWW, [Ge—Si—O]-MWW, [In—Si—O]-MWW, [Pb—Si—O]-MWW, [Cu—Si—O]-MWW and [Ti—Si—O]-MWW, including mixtures of two or more thereof, wherein preferably the zeolite is selected from the group consisting of MCM-22, ITQ-1, SSZ-25, [B—Si—O]-MWW, [Sn—Si—O]-MWW, [AI-Si—O]-MWW, [Zr—Si—O]-MWW, [Ge—Si—O]-MWW, [Zn—Si—O]-MWW, [Ga—Si—O]-MWW, [Pb—Si—O]-MWW, [Cu—Si—O]-MWW and [Ti—Si—O]-MWW, including mixtures of two or more thereof, preferably from the group consisting of MCM-22, ITQ-1, SSZ-25, [B—Si—O]-MWW, [Sn—Si—O]-MWW and [Ti—Si—O]-MWW, more preferably from the group consisting of [B—SiO]-MWW, [Sn—Si—O]-MWW, and [Ti—Si—O]-MWW, including mixtures of two or more thereof, wherein more preferably the zeolite in (2) is a [B—Si—O]-MWW and/or a [Sn—Si—O]-MWW, wherein more preferably the zeolite in (2) is a [B—Si—O]-MWW.

It is preferred that the zeolite in (2) comprises, preferably consists of, a [B—Si—O]-MWW zeolite, wherein the [B—Si—O]-MWW zeolite has a boron content in the range of from 0.1 to 5 wt.-% B calculated as the element and based on 100 wt.-% of B, Si, and O contained in the zeolite, preferably from 0.3 to 3.5 wt.-%, more preferably from 0.5 to 2.8 wt.-%, more preferably from 1 to 2.3 wt.-%, more preferably from 1.1 to 1.9 wt.-%, more preferably from 1.2 to 1.7 wt.-%, and more preferably from 1.3 to 1.5 wt.-%.

It is preferred that the zeolite in (2) comprises, preferably consists of, a [B—Si—O]-MWW zeolite, wherein the [B—Si—O]-MWW zeolite has been deboronated, wherein preferably from 50 to 99.9 wt.-% of boron initially contained in the zeolite has been removed from the zeolite based on 100 wt.-% of boron initially contained in the [B—Si—O]-MWW zeolite, wherein more preferably from 75 to 99 wt.-% of boron initially contained in the zeolite has been removed, more preferably from 85 to 98 wt.-%, more preferably from 90 to 97 wt.-%, more preferably from 94 to 96 wt.-%. In cases in which the [B—Si—O]-MWW zeolite has been deboronated, it is further preferred that prior to the deboronation, the [B—Si—O]-MWW zeolite initially contained from 0.1 to 5 wt.-% B calculated as the element and based on 100 wt.-% of B, Si, and O contained in the zeolite, preferably from 0.5 to 3 wt.-%, more preferably from 0.8 to 2 wt.-%, more preferably from 1 to 1.8 wt.-%, more preferably from 1.1 to 1.6 wt.-%, more preferably from 1.2 to 1.4 wt.-%.

It is preferred that the zeolite in (2) comprises, preferably consists of, a [Ti—Si—O]-MWW zeolite, wherein the [Ti—Si—O]-MWW zeolite has a titanium content in the range of from 0.1 to 5 wt.-% Ti calculated as the element and based on 100 wt.-% of Ti, Si, and O contained in the zeolite, preferably from 0.5 to 3.5 wt.-%, more preferably from 1 to 3 wt.-%, more preferably from 1.4 to 2.5 wt.-%, more preferably from 1.6 to 2.2 wt.-%, more preferably from 1.8 to 2 wt.-%.

It is preferred that the zeolite in (2) comprises, preferably consists of, a [Sn—Si—O]-MWW zeolite, wherein the [Sn—Si—O]-MWW zeolite has a tin content in the range of from 0.1 to 5 wt.-% Sn calculated as the element and based on 100 wt.-% of Sn, Si, and O contained in the zeolite, preferably from 0.5 to 4 wt.-%, more preferably from 1 to 3.5 wt.-%, more preferably from 1.3 to 3 wt.-%, more preferably from 1.5 to 2.8 wt.-%, more preferably from 1.8 to 2.5 wt.-%, more preferably from 2 to 2.2 wt.-%.

It is preferred that the acidity of the zeolite provided in (2) as determined by NH₃-TPD is in the range of from 0.05 to 3.5 mmol/g, preferably from 0.1 to 3.0 mmol/g, more preferably from 0.15 to 2.5 mmol/g, more preferably from 0.18 to 2.0 mmol/g, wherein the NH₃-TPD is preferably determined according to Reference Example 1.

It is preferred that the acidity of the zeolite provided in (2) as determined by NH₃-TPD is in the range of from 0.01 to 0.400 mmol/g, preferably from 0.05 to 0.35 mmol/g, more preferably from 0.1 to 0.30 mmol/g, more preferably from 0.15 to 0.25 mmol/g, more preferably from 0.18 to 0.20 mmol/g, wherein the NH₃-TPD is preferably determined according to Reference Example 1

It is preferred that in (2) the zeolite contains phosphorous, wherein preferably, the zeolite contains phosphorous in an amount ranging from 0.01 to 5 wt.-% based on 100 wt.-% of Si and O contained in the zeolite, more preferably from 0.05 to 3 wt.-%, more preferably from 0.1 to 1.5 wt.-%, more preferably from 0.3 to 1 wt.-%, more preferably from 0.4 to 0.8 wt.-%, more preferably from 0.5 to 0.7 wt.-%.

It is preferred that in (2) the catalyst is provided as a shaped body, wherein preferably the body is shaped by extrusion. Further it is preferred that the shaped body contains a binder, wherein the binder preferably comprises, preferably consists of, at least one metal oxide selected from the group consisting of SiO₂, and ZrO₂, including mixtures thereof, wherein more preferably the binder comprises, preferably consists of, SiO₂. Yet further, it is preferred that the acidity of the shaped body as determined by NH₃-TPD is in the range of from 0.001 to 0.400 mmol/g, preferably from 0.05 to 0.35 mmol/g, more preferably from 0.1 to 0.30 mmol/g, more preferably from 0.15 to 0.25 mmol/g, more preferably from 0.18 to 0.20 mmol/g, wherein the NH₃-TPD is preferably determined according to Reference Example 1. Yet further, it is preferred that the binder is substantially free of Al₂O₃.

Within the meaning of the present invention, the term “substantially free” with regard to an element or compound(s) indicates that said element or compound(s) is present in an amount of 1 wt.-% or less calculated as the element or compound(s) and based on 100 wt.-% of the entity which is indicated as being substantially free thereof, preferably in an amount of 0.5 wt.-% or less, more preferably of 0.1 wt.-% or less, more preferably of 0.05 wt.-% or less, more preferably of 0.01 wt.-% or less, more preferably of 0.005 wt.-% or less, and more preferably of 0.001 wt.-% or less.

It is preferred that the catalyst is substantially free of Al₂O₃.

It is preferred that the 1,1-diarylethane provided in (1) and contacted with the catalyst in (3) is in the gas phase.

Further in the case where 1,1-diarylethane provided in (1) and contracted with the catalyst in (3) is in the gas phase, it is preferred that the gas hourly space velocity at which the feed is contacted with the catalyst in (3) is in the range of 150 to 500 h⁻¹, preferably from 200 to 350 h⁻¹.

It is preferred that the feed in (1) further comprises an inert gas, wherein the inert gas is preferably selected from the group consisting of N₂, CO₂, noble gases, and mixtures thereof, wherein more preferably the inert gas comprises, preferably consists of, N₂ and/or Ar, more preferably N₂. Yet further, it is preferred that the feed in (1), excluding the inert gas, contains a ratio to the catalyst ranging from 0.05 to 0.5 g/(g h), preferably from 0.1 to 0.2 g/(g h).

Alternatively, it is preferred that the mass hourly space velocity at which the feed, wherein the mass hourly space velocity at which the feed, is contacted with the catalyst in (3) is in the range of 0.05 to 0.5 g/(g h), more preferably from 0.1 to 0.2 g/(g h), wherein the feed preferably does not contain an inert gas, wherein more preferably the feed does not contain an inert gas according to claim 20.

It is preferred that the feed in (1) further contains H₂O. Yet further, it is preferred that the feed in (1) displays an H₂O: 1,1-diarylethane weight ratio in the range of from 0.3 to 3, preferably from 0.5 to 2.5, more preferably from 0.8 to 1.5, more preferably from 1 to 1.2.

It is preferred that in (3) the reaction occurs in a fixed bed reactor or in a fluidized bed reactor, preferably in a fixed bed reactor.

It is preferred that contacting in (3) is conducted at a pressure in the range of from 0.9 to 1.3 bara, preferably from 1 to 1.1 bara.

It is preferred that contacting in (3) is conducted at a temperature in the range of from 250 to 600 °C, preferably from 300 to 500 °C, more preferably from 330 to 450 °C, more preferably from 350 to 430 °C, more preferably from 360 to 410 °C, more preferably from 370 to 390 °C.

It is preferred that the process includes a step of regenerating the catalyst provided in (2), and contacted with the feed in (3), at regular intervals, wherein the catalyst is regenerated by calcination at a temperature in the range of from 300 to 850 °C, preferably from 350 to 700 °C, more preferably from 400 to 600 °C, more preferably from 450 to 550 °C. Yet further, it is preferred that the calcination is conducted for a period ranging from 1 to 24 h, more preferably from 2 to 12 h, more preferably from 3 to 8 h.

It is preferred that unreacted 1,1-diarylethane obtained in (3) and/or (4) is recycled to (1), preferably after separation from the reaction product obtained in (3) and/or after separation from the residual product obtained in (4) after separation of the alkylstyrene.

It is preferred that the 1,1-diarylethane compound provided in (1) is obtained from a process comprising the acid catalyzed reaction of an alkylbenzene and an acetaldehyde. Further it is preferred that the acid catalyst is sulfuric acid. Furthermore and independently thereof, it is preferred that the alkyl group of the alkylbenzene is selected from the group consisting of C1-C10 alkyl, preferably C2 to C6 alkyl, more preferably C3 to C5 alkyl, and more preferably C4 alkyl, wherein more preferably the alkyl group of the alkylbenzene is selected from the group consisting of ethyl, n-propyl, i-propyl, n-butyl, i-butyl, and 1-methylpropyl, more preferably from the group consisting of i-propyl, n-butyl, i-butyl, and 1-methylpropyl, more preferably from the group consisting of i-propyl, i-butyl, and 1-methylpropyl, wherein more preferably the alkyl group of the alkylbenzene is i-butyl. Yet further, it is particularly preferred that the alkylbenzene is isobutylbenzene.

It is preferred that the alkylbenzene obtained in (3) and/or (4) is be recycled to the acid catalyzed reaction, preferably after separation from the reaction product obtained in (3) and/or after separation from the residual product obtained in (4) after separation of the alkylstyrene.

It is preferred that the carbonylation catalyst provided in (6) comprises a metal selected from the group consisting of Pd, Rh, Ir, Ru, Ni, Co, and Fe, including mixtures of two or more thereof, wherein preferably the platinum group metal comprises Pd, wherein more preferably the metal is Pd.

It is preferred that contacting in (7) is conducted at a temperature in the range of from 60 to 150 °C, preferably from 80 to 130 °C.

It is preferred that contacting in (7) is conducted at a pressure in the range of from 50 to 150 bara, preferably from 55 to 100 bara.

It is preferred that the process does not involve the use of HF, wherein preferably the process does not involve the use of a fluoride or of a fluorine containing compound.

It is preferred that the process does not involve the use of AC₃, wherein preferably the process does not involve the use of AlX₃, wherein X stands for CI or Br, wherein more preferably the process does not involve the use of an aluminum containing compound.

It is preferred that the feed in (1) is a feed stream, and the catalytic cracking in (3) is conducted as a continuous process.

It is preferred that in (2) the zeolite having the MWW-type framework structure is prepared from a process comprising
(a) hydrothermally synthesizing an MWW precursor from a synthesis mixture containing at least one silicon source, preferably ammonia stabilized colloidal silica, at least one boron source, preferably boric acid, and at least one template compound, preferably selected from the group consisting of piperidine, hexamethylene imine, and a mixture thereof, to obtain the MWW precursor in its mother liquor;
(b) separating the MWW precursor from its mother liquor;
(c) optionally drying the MWW precursor separated according to (b);
(d) calcining the MWW precursor obtained from (b) or (c), preferably at a temperature in the range of from 500 to 700 °C, and obtaining a [B—Si—O]-MWW.

Alternatively, it is preferred that in (2) the zeolite having the MWW framework type is prepared from a process comprising
(i) preparing a [B—Si—O]-MWW according to a process comprising steps (a) to (d) as defined in claim 43;
(ii) deboronating the [B—Si—O]-MWW by treating the [B—Si—O]-MWW with a liquid solvent system thereby obtaining a deboronated zeolite having the MWW-type framework structure; wherein the liquid solvent system is selected from the group consisting of water, monohydric alcohols, polyhydric alcohols, and mixtures of two or more thereof, and wherein said liquid solvent system does not contain an inorganic or organic acid or a salt thereof, the acid being selected from the group consisting of hydrochloric acid, sulfuric acid, nitric acid, phosphoric acid, formic acid, acetic acid, propionic acid, oxalic acid, and tartaric acid.

In cases in which in (2) the zeolite having the MWW framework type is prepared from a process comprising steps (a) to (d) or (i) to (ii), it is preferred that the liquid solvent system does not contain an inorganic or organic acid, or a salt thereof. Yet further, it is preferred that the liquid solvent system is selected from the group consisting of water, methanol, ethanol, propanol, ethane-1,2-diol, propane-1,2-diol, propane-1,3-diol, propane-1,2,3-triol, and mixtures of two or more thereof, the liquid solvent system preferably being water.

In cases in which in (2) the zeolite having the MWW framework type is prepared from a process comprising steps (i) to (ii), it is preferred that the treating according to (ii) is carried out at a temperature in the range of from 50 to 125 °C. Yet further, it is preferred that the treating according to (ii) is carried out for a time in the range of from 6 to 20 h. Yet further, it is preferred that the treating according to (ii) is carried out in at least 2 separate steps, wherein between at least 2 treating steps, the deboronated zeolite having the MWW-type framework structure is dried, preferably at a temperature in the range of from 100 to 150 °C.

Furthermore and independently thereof, in cases in which in (2) the zeolite having the MWW framework type is prepared from a process comprising steps (i) to (ii), it is preferred that the process further comprises
(iii) post-treating the deboronated zeolite having the MWW-type framework structure obtained from (ii) by a process comprising
(iii.1) separating the deboronated zeolite having the MWW-type framework structure from the liquid solvent system;
(iii.2) optionally drying the separated deboronated zeolite having the MWW-type framework structure, preferably by spray-drying;
(iii.3) optionally calcining the deboronated zeolite having the MWW-type framework structure obtained from (iii.1) or (iii.2), preferably at a temperature in the range of from 500 to 700 °C.

Yet further, in cases in which in (2) the zeolite having the MWW framework type is prepared from a process comprising steps (i) to (ii), preferably (i) to (iii), it is preferred that the process further comprises
(iv) incorporating at least one heteroatom into the deboronated zeolite having the MWW-type framework structure thereby obtaining a zeolite having the MWW-type structure containing at least one heteroatom by a process comprising
(iv.1) preparing a synthesis mixture containing the deboronated zeolite having the MWW-type framework structure obtained according to (ii) or (iii), preferably (iii), a template compound, preferably selected from the group consisting of piperidine, hexamethylene imine, and a mixture thereof, and at least one source of at least one heteroatom, wherein the heteroatom is preferably selected from the group consisting of Ti, Sn, Al, Zr, V, Nb, Ta, Cr, Mo, W, Mn, Fe, Co, Ni, Zn, Ga, Ge, In, Cu, and Pb, including mixtures of two or more thereof, wherein more preferably the heteroatom is Al, Zr, Ge, Zn, Ga, Pb, Cu, Ti and/or Sn, more preferably Ti and/or Sn;
(iv.2) hydrothermally synthesizing the zeolite having the MWW-type structure containing at least one heteroatom from the synthesis mixture obtained from (iv.1) thereby obtaining the zeolite having the MWW-type structure containing at least one heteroatom in its mother liquor;
(iv.3) separating the zeolite having the MWW-type structure containing at least one heteroatom from its mother liquor;
(iv.4) optionally drying the zeolite having the MWW-type structure containing at least one heteroatom separated according to (iv.3), preferably by spray-drying;
(iv.5) optionally calcining the zeolite having the MWW-type structure containing at least one heteroatom obtained from (iv.3) or (iv.4), preferably at a temperature in the range of from 500 to 700 °C.

The present invention is further illustrated by the following set of embodiments and combinations of embodiments resulting from the dependencies and back-references as indicated. In particular, it is noted that in each instance where a range of embodiments is mentioned, for example in the context of a term such as “The process according to any one of embodiments 1 to 4”, every embodiment in this range is meant to be explicitly disclosed for the skilled person i.e. the wording of this term is to be understood by the skilled person as being synonymous to “The process according to any one of embodiments 1, 2, 3 and 4”. Further, it is explicitly noted that the following set of embodiments is not the set of claims determining the extent of protection, but represents a suitably structured part of the description directed to general and preferred aspects of the present invention.
1. A process for the production of an alkylstyrene comprising
   (1) providing a feed containing a 1,1-diarylethane compound, wherein the aryl-group is an alkylphenyl group;
   (2) providing a catalyst comprising a zeolite having the MWW-type framework structure;
   (3) contacting the feed provided in (1) with the catalyst provided in (2) for the catalytic cracking of the 1,1-diarylethane to the alkylstyrene and the alkylbenzene;
   (4) separating the alkylstyrene from the reaction product obtained in (3).
2. A process for the production of an alkylphenyl propionic acid comprising
   (1) providing a feed containing a 1,1-diarylethane compound, wherein the aryl-group is an alkylphenyl group;
   (2) providing a catalyst comprising a zeolite having the MWW-type framework structure;
   (3) contacting the feed provided in (1) with the catalyst provided in (2) for the catalytic cracking of the 1,1-diarylethane to the alkylstyrene and the alkylbenzene;
   (4) optionally separating the alkylstyrene from the reaction product obtained in (3);
   (5) preparing a mixture containing CO, the alkylstyrene-containing reaction product obtained in (3) or the alkylstyrene obtained in (4), and optionally a solvent system;
   (6) providing a carbonylation catalyst;
   (7) contacting the mixture obtained in (5) with the carbonylation catalyst provided in (6) for converting the alkylstyrene to the alkylphenypropionic acid.
3. The process according to embodiment 1 or 2, wherein in (1) the alkyl group of the alkylphenyl group is selected from the group consisting of C1-C10 alkyl, preferably C2 to C6 alkyl, more preferably C3 to C5 alkyl, and more preferably C4 alkyl, wherein more preferably the alkyl group of the alkylphenyl group is selected from the group consisting of ethyl, n-propyl, i-propyl, n-butyl, i-butyl, and 1-methylpropyl, more preferably from the group consisting of i-propyl, n-butyl, i-butyl, and 1-methylpropyl, more preferably from the group consisting of i-propyl, i-butyl, and 1-methylpropyl, wherein more preferably the alkyl group of the alkylphenyl group is i-butyl.
4. The process according to any of embodiments 1 to 3, wherein in (1) the alkylphenyl group is selected from the group consisting of o-isobutylphenyl, m-isobutylphenyl, and p-isobutylphenyl, wherein preferably the alkylphenyl group is p-isobutylphenyl.
5. The process according to any of embodiments 1 to 4, wherein the zeolite in (2) is selected from the group consisting of PSH-3, SSZ-25, ERB-1, MCM-22, ITQ-1, SSZ-25, [Ga—Si—O]-MWW, [B—Si—O]-MWW, [Sn—Si—O]-MWW, [AI-Si—O]-MWW, [Zr—Si—O]-MWW, [V—Si—O]-MWW, [Nb—Si—O]-MWW, [Ta—Si—O]-MWW, [Cr—Si—O]-MWW, [Mo—Si—O]-MWW, [W—Si—O]-MWW, [Mn—Si—O]-MWW, [Fe—Si—O]-MWW, [Co—Si—O]-MWW, [Ni—Si—O]-MWW, [Zn—Si—O]-MWW, [Ga—Si—O]-MWW, [Ge—Si—O]-MWW, [In—Si—O]-MWW, [Pb—Si—O]-MWW, [Cu—Si—O]-MWW and [Ti—Si—O]-MWW, including mixtures of two or more thereof, wherein preferably the zeolite is selected from the group consisting of MCM-22, ITQ-1, SSZ-25, [B—Si—O]-MWW, [Sn—SiO]-MWW, [AI-Si—O]-MWW, [Zr—Si—O]-MWW, [Ge—Si—O]-MWW, [Zn—Si—O]-MWW, [Ga—Si—O]-MWW, [Pb—Si—O]-MWW, [Cu—Si—O]-MWW and [Ti—Si—O]-MWW, including mixtures of two or more thereof, preferably from the group consisting of MCM-22, ITQ-1, SSZ-25, [B—Si—O]-MWW, [Sn—Si—O]-MWW and [Ti—Si—O]-MWW, more preferably from the group consisting of [B—Si—O]-MWW, [Sn—Si—O]-MWW, and [Ti—Si—O]-MWW, including mixtures of two or more thereof, wherein more preferably the zeolite in (2) is a [B—Si—O]-MWW and/or a [Sn—Si—O]-MWW, wherein more preferably the zeolite in (2) is a [B—Si—O]-MWW.
6. The process according to any of embodiments 1 to 5, wherein the zeolite in (2) comprises, preferably consists of, a [B—Si—O]-MWW zeolite, wherein the [B—Si—O]-MWW zeolite has a boron content in the range of from 0.1 to 5 wt.-% B calculated as the element and based on 100 wt.-% of B, Si, and O contained in the zeolite, preferably from 0.3 to 3.5 wt.-%, more preferably from 0.5 to 2.8 wt.-%, more preferably from 1 to 2.3 wt.-%, more preferably from 1.1 to 1.9 wt.-%, more preferably from 1.2 to 1.7 wt.-%, and more preferably from 1.3 to 1.5 wt.-%.
7. The process according to any of embodiments 1 to 6, wherein the zeolite in (2) comprises, preferably consists of, a [B—Si—O]-MWW zeolite, wherein the [B—Si—O]-MWW zeolite has been deboronated, wherein preferably from 50 to 99.9 wt.-% of boron initially contained in the zeolite has been removed from the zeolite based on 100 wt.-% of boron initially contained in the [B—Si—O]-MWW zeolite, wherein more preferably from 75 to 99 wt.-% of boron initially contained in the zeolite has been removed, more preferably from 85 to 98 wt.-%, more preferably from 90 to 97 wt.-%, more preferably from 94 to 96 wt.-%.
8. The process according to embodiment 7, wherein prior to the deboronation, the [B—Si—O]-MWW zeolite initially contained from 0.1 to 5 wt.-% B calculated as the element and based on 100 wt.-% of B, Si, and O contained in the zeolite, preferably from 0.5 to 3 wt.-%, more preferably from 0.8 to 2 wt.-%, more preferably from 1 to 1.8 wt.-%, more preferably from 1.1 to 1.6 wt.-%, more preferably from 1.2 to 1.4 wt.-%.
9. The process according to any of embodiments 1 to 8, wherein the zeolite in (2) comprises, preferably consists of, a [Ti—Si—O]-MWW zeolite, wherein the [Ti—Si—O]-MWW zeolite has a titanium content in the range of from 0.1 to 5 wt.-% Ti calculated as the element and based on 100 wt.-% of Ti, Si, and O contained in the zeolite, preferably from 0.5 to 3.5 wt.-%, more preferably from 1 to 3 wt.-%, more preferably from 1.4 to 2.5 wt.-%, more preferably from 1.6 to 2.2 wt.-%, more preferably from 1.8 to 2 wt.-%.
10. The process according to any of embodiments 1 to 9, wherein the zeolite in (2) comprises, preferably consists of, a [Sn—Si—O]-MWW zeolite, wherein the [Sn—Si—O]-MWW zeolite has a tin content in the range of from 0.1 to 5 wt.-% Sn calculated as the element and based on 100 wt.-% of Sn, Si, and O contained in the zeolite, preferably from 0.5 to 4 wt.-%, more preferably from 1 to 3.5 wt.-%, more preferably from 1.3 to 3 wt.-%, more preferably from 1.5 to 2.8 wt.-%, more preferably from 1.8 to 2.5 wt.-%, more preferably from 2 to 2.2 wt.-%.
11. The process according to any of embodiments 1 to 10, wherein the acidity of the zeolite provided in (2) as determined by NH₃-TPD is in the range of from 0.05 to 3.5 mmol/g, preferably from 0.1 to 3.0 mmol/g, more preferably from 0.15 to 2.5 mmol/g, more preferably from 0.18 to 2.0 mmol/g, wherein the NH₃-TPD is preferably determined according to Reference Example 1.
12. The process according to any of embodiments 1 to 11, wherein the acidity of the zeolite provided in (2) as determined by NH₃-TPD is in the range of from 0.01 to 0.400 mmol/g, preferably from 0.05 to 0.35 mmol/g, more preferably from 0.1 to 0.30 mmol/g, more preferably from 0.15 to 0.25 mmol/g, more preferably from 0.18 to 0.20 mmol/g, wherein the NH₃-TPD is preferably determined according to Reference Example 1.
13. The process according to any of embodiments 1 to 12, wherein in (2) the zeolite contains phosphorous, wherein preferably, the zeolite contains phosphorous in an amount ranging from 0.01 to 5 wt.-% based on 100 wt.-% of Si and O contained in the zeolite, more preferably from 0.05 to 3 wt.-%, more preferably from 0.1 to 1.5 wt.-%, more preferably from 0.3 to 1 wt.-%, more preferably from 0.4 to 0.8 wt.-%, more preferably from 0.5 to 0.7 wt.-%.
14. The process according to any of embodiments 1 to 13, wherein in (2) the catalyst is provided as a shaped body, wherein preferably the body is shaped by extrusion.
15. The process according to embodiment 14, wherein the shaped body contains a binder, wherein the binder preferably comprises, preferably consists of, at least one metal oxide selected from the group consisting of SiO₂, and ZrO₂, including mixtures thereof, wherein more preferably the binder comprises, preferably consists of, SiO₂.
16. The process according to embodiment 14 or 15, wherein the acidity of the shaped body as determined by NH₃-TPD is in the range of from 0.001 to 0.400 mmol/g, preferably from 0.05 to 0.35 mmol/g, more preferably from 0.1 to 0.30 mmol/g, more preferably from 0.15 to 0.25 mmol/g, more preferably from 0.18 to 0.20 mmol/g, wherein the NH₃-TPD is preferably determined according to Reference Example 1.
17. The process according to embodiment 15 or 16, wherein the binder is substantially free of Al₂O₃.
18. The process according to any of embodiments 1 to 17, wherein the catalyst is substantially free of Al₂O₃.
19. The process according to any of embodiments 1 to 18, wherein the 1,1-diarylethane provided in (1) and contacted with the catalyst in (3) is in the gas phase.
20. The process according to embodiment 19, wherein the gas hourly space velocity at which the feed is contacted with the catalyst in (3) is in the range of 150 to 500 h⁻¹, preferably from 200 to 350 h⁻¹.
21. The process according to any of embodiments 1 to 20, wherein the feed in (1) further comprises an inert gas, wherein the inert gas is preferably selected from the group consisting of N₂, CO₂, noble gases, and mixtures thereof, wherein more preferably the inert gas comprises, preferably consists of, N₂ and/or Ar, preferably N₂.
22. The process according to embodiment 21, wherein the feed in (1), excluding the inert gas, contains a ratio to the catalyst ranging from 0.05 to 0.5 g/(g h), preferably from 0.1 to 0.2 g/(g h).
23. The process according to any of embodiments 1 to 21, wherein the mass hourly space velocity at which the feed, is contacted with the catalyst in (3) is in the range of 0.05 to 0.5 g/(g h), more preferably from 0.1 to 0.2 g/(g h), wherein the feed preferably does not contain an inert gas, wherein more preferably the feed does not contain an inert gas according to claim 20.
24. The process according to any of embodiments 1 to 23, wherein the feed in (1) further contains H₂O.
25. The process according to embodiment 24, wherein the feed in (1) displays an H₂O: 1,1-diarylethane weight ratio in the range of from 0.3 to 3, more preferably from 0.5 to 2.5, more preferably from 0.8 to 1.5, more preferably from 1 to 1.2.
26. The process according to any of embodiments 1 to 25, wherein in (3) the reaction occurs in a fixed bed reactor or in a fluidized bed reactor, preferably in a fixed bed reactor.
27. The process according to any of embodiments 1 to 26, wherein contacting in (3) is conducted at a pressure in the range of from 0.9 to 1.3 bara, preferably from 1 to 1.1 bara.
28. The process according to any of embodiments 1 to 27, wherein contacting in (3) is conducted at a temperature in the range of from 250 to 600 °C, preferably from 300 to 500 °C, more preferably from 330 to 450 °C, more preferably from 350 to 430 °C, more preferably from 360 to 410 °C, more preferably from 370 to 390 °C.
29. The process according to any of embodiments 1 to 28, wherein the process includes a step of regenerating the catalyst provided in (2), and contacted with the feed in (3), at regular intervals, wherein the catalyst is regenerated by calcination at a temperature in the range of from 300 to 850 °C, preferably from 350 to 700 °C, more preferably from 400 to 600 °C, more preferably from 450 to 550 °C.
30. The process according to embodiment 29, wherein the calcination is conducted for a period ranging from 1 to 24 h, more preferably from 2 to 12 h, more preferably from 3 to 8 h.
31. The process according to any of embodiments 1 to 30, wherein unreacted 1,1-diarylethane obtained in (3) and/or (4) is recycled to (1), preferably after separation from the reaction product obtained in (3) and/or after separation from the residual product obtained in (4) after separation of the alkylstyrene.
32. The process according to any of embodiments 1 to 31, wherein the 1,1-diarylethane compound provided in (1) is obtained from a process comprising the acid catalyzed reaction of an alkylbenzene and an acetaldehyde.
33. The method according to embodiment 32, wherein the acid catalyst is sulfuric acid.
34. The process according to embodiment 32 or 33, wherein in the alkyl group of the alkylbenzene is selected from the group consisting of C1-C10 alkyl, preferably C2 to C6 alkyl, more preferably C3 to C5 alkyl, and more preferably C4 alkyl, wherein more preferably the alkyl group of the alkylbenzene is selected from the group consisting of ethyl, n-propyl, ipropyl, n-butyl, i-butyl, and 1-methylpropyl, more preferably from the group consisting of ipropyl, n-butyl, i-butyl, and 1-methylpropyl, more preferably from the group consisting of ipropyl, i-butyl, and 1-methylpropyl, wherein more preferably the alkyl group of the alkylbenzene is i-butyl.
35. The process according to any embodiments 32 to 34, wherein the alkylbenzene is isobutylbenzene.
36. The process according to any of embodiments 1 to 35, wherein the alkylbenzene obtained in (3) and/or (4) is be recycled to the acid catalyzed reaction, preferably after separation from the reaction product obtained in (3) and/or after separation from the residual product obtained in (4) after separation of the alkylstyrene.
37. The process according to any of embodiments 2 to 36, wherein the carbonylation catalyst provided in (6) comprises a metal selected from the group consisting of Pd, Rh, Ir, Ru, Ni, Co, and Fe, including mixtures of two or more thereof, wherein preferably the platinum group metal comprises Pd, wherein more preferably the platinum group metal is Pd.
38. The process according to any of embodiments 2 to 37, wherein contacting in (7) is conducted at a temperature in the range of from 60 to 150 °C, preferably from 80 to 130 °C.
39. The process according to any of embodiments 2 to 38, wherein contacting in (7) is conducted at a pressure in the range of from 50 to 150 bara, preferably from 55 to 100 bara.
40. The process according to any of embodiments 1 to 39, wherein the process does not involve the use of HF, wherein preferably the process does not involve the use of a fluoride or of a fluorine containing compound.
41. The process according to any of embodiments 1 to 40, wherein the process does not involve the use of AlCl₃, wherein preferably the process does not involve the use of AlX₃, wherein X stands for CI or Br, wherein more preferably the process does not involve the use of an aluminum containing compound.
42. The process according to any of embodiments 1 to 41, wherein the feed in (1) is a feed stream, and the catalytic cracking in (3) is conducted as a continuous process.
43. The process according to any of embodiments 1 to 42, wherein in (2) the zeolite having the MWW-type framework structure is prepared from a process comprising
   (a) hydrothermally synthesizing an MWW precursor from a synthesis mixture containing at least one silicon source, preferably ammonia stabilized colloidal silica, at least one boron source, preferably boric acid, and at least one template compound, preferably selected from the group consisting of piperidine, hexamethylene imine, and a mixture thereof, to obtain the MWW precursor in its mother liquor;
   (b) separating the MWW precursor from its mother liquor;
   (c) optionally drying the MWW precursor separated according to (b);
   (d) calcining the MWW precursor obtained from (b) or (c), preferably at a temperature in the range of from 500 to 700 °C, and obtaining a [B—Si—O]-MWW.
44. The process according to any of embodiments 1 to 42, wherein in (2) the zeolite having the MWW framework type is prepared from a process comprising
   (i) preparing a [B—Si—O]-MWW according to a process comprising steps (a) to (d) as defined in embodiment 43;
   (ii) deboronating the [B—Si—O]-MWW by treating the [B—Si—O]-MWW with a liquid solvent system thereby obtaining a deboronated zeolite having the MWW-type framework structure;
   wherein the liquid solvent system is selected from the group consisting of water, monohydric alcohols, polyhydric alcohols, and mixtures of two or more thereof, and wherein said liquid solvent system does not contain an inorganic or organic acid or a salt thereof, the acid being selected from the group consisting of hydrochloric acid, sulfuric acid, nitric acid, phosphoric acid, formic acid, acetic acid, propionic acid, oxalic acid, and tartaric acid.
45. The process according to embodiment 43 or 44, wherein the liquid solvent system does not contain an inorganic or organic acid, or a salt thereof.
46. The process according to any of embodiments 43 to 45, wherein the liquid solvent system is selected from the group consisting of water, methanol, ethanol, propanol, ethane-1,2-diol, propane-1,2-diol, propane-1,3-diol, propane-1,2,3-triol, and mixtures of two or more thereof, the liquid solvent system preferably being water.
47. The process according to any of embodiments 44 to 46, wherein the treating according to (ii) is carried out at a temperature in the range of from 50 to 125 °C.
48. The process according to any of embodiments 44 to 47, wherein the treating according to (ii) is carried out for a time in the range of from 6 to 20 h.
49. The process according to any of embodiments 44 to 48, wherein the treating according to (ii) is carried out in at least 2 separate steps, wherein between at least 2 treating steps, the deboronated zeolite having the MWW-type framework structure is dried, preferably at a temperature in the range of from 100 to 150 °C.
50. The process according to any of embodiments 44 to 49 further comprising
   (iii) post-treating the deboronated zeolite having the MWW-type framework structure obtained from (ii) by a process comprising
   (iii.1) separating the deboronated zeolite having the MWW-type framework structure from the liquid solvent system;
   (iii.2) optionally drying the separated deboronated zeolite having the MWW-type framework structure, preferably by spray-drying;
   (iii.3) optionally calcining the deboronated zeolite having the MWW-type framework structure obtained from (iii. 1) or (iii.2), preferably at a temperature in the range of from 500 to 700 °C.
51. The process according to any of embodiments 44 to 50, preferably according to claim 50, further comprising
   (iv) incorporating at least one heteroatom into the deboronated zeolite having the MWW-type framework structure thereby obtaining a zeolite having the MWW-type structure containing at least one heteroatom by a process comprising
   (iv.1) preparing a synthesis mixture containing the deboronated zeolite having the MWW-type framework structure obtained according to (ii) or (iii), preferably (iii), a template compound, preferably selected from the group consisting of piperidine, hexamethylene imine, and a mixture thereof, and at least one source of at least one heteroatom, wherein the heteroatom is preferably selected from the group consisting of Ti, Sn, Al, Zr, V, Nb, Ta, Cr, Mo, W, Mn, Fe, Co, Ni, Zn, Ga, Ge, In, Cu, and Pb, including mixtures of two or more thereof, wherein more preferably the heteroatom is Al, Zr, Ge, Zn, Ga, Pb, Cu, Ti and/or Sn, more preferably Ti and/or Sn;
   (iv.2) hydrothermally synthesizing the zeolite having the MWW-type structure containing at least one heteroatom from the synthesis mixture obtained from (iv.1) thereby obtaining the zeolite having the MWW-type structure containing at least one heteroatom in its mother liquor;
   (iv.3) separating the zeolite having the MWW-type structure containing at least one heteroatom from its mother liquor;
   (iv.4) optionally drying the zeolite having the MWW-type structure containing at least one heteroatom separated according to (iv.3), preferably by spray-drying;
   (iv.5) optionally calcining the zeolite having the MWW-type structure containing at least one heteroatom obtained from (iv.3) or (iv.4), preferably at a temperature in the range of from 500 to 700 °C.

The present invention is further illustrated by the following examples and comparative examples.

### EXAMPLES

### Reference Example 1: Determination of the Acid Sites: Temperature Programmed Desorption of Ammonia (NH₃-TPD)

The temperature-programmed desorption of ammonia (NH₃-TPD) was conducted in an automated chemisorption analysis unit (Micromeritics AutoChem II 2920) having a thermal conductivity detector. Continuous analysis of the desorbed species was accomplished using an online mass spectrometer (OmniStar QMG200 from Pfeiffer Vacuum). The sample (0.1 g) was introduced into a quartz tube and analysed using the program described below. The temperature was measured by means of a Ni/Cr/Ni thermocouple immediately above the sample in the quartz tube. For the analyses, He of purity 5.0 was used. Before any measurement, a blank sample was analysed for calibration.
1. Preparation: Commencement of recording; one measurement per second. Wait for 10 minutes at 25 °C and a He flow rate of 30 cm³/min (room temperature (about 25 °C) and 1 atm); heat up to 600 °C at a heating rate of 20 K/min; hold for 10 minutes. Cool down under a He flow (30 cm³/min) to 100 °C at a cooling rate of 20 K/min (furnace ramp temperature); Cool down under a He flow (30 cm³/min) to 100 °C at a cooling rate of 3 K/min (sample ramp temperature).
2. Saturation with NH₃: Commencement of recording; one measurement per second. Change the gas flow to a mixture of 10% NH₃ in He (75 cm³/min; 100 °C and 1 atm) at 100 °C; hold for 30 min.
3. Removal of the excess: Commencement of recording; one measurement per second. Change the gas flow to a He flow of 75 cm³/min (100 °C and 1 atm) at 100 °C; hold for 60 min.
4. NH₃-TPD: Commencement of recording; one measurement per second. Heat up under a He flow (flow rate: 30 cm³/min) to 600 °C at a heating rate of 10 K/min; hold for 30 min.
5. End of measurement.

Desorbed ammonia was measured by means of the online mass spectrometer, which demonstrated that the signal from the thermal conductivity detector was caused by desorbed ammonia. This involved utilizing the m/z=16 signal from ammonia in order to monitor the desorption of the ammonia. The amount of ammonia adsorbed (mmol/g of sample) was ascertained by means of the Micromeritics software through integration of the TPD signal with a horizontal baseline.

### Reference Example 2: Preparation of B-MWW Catalysts

B-MWW (1.4 wt.-% B) powder was produced based on example 1.1 of WO 2014/122152 A. 50.00 g of the obtained B-MWW powder was respectively put into a kneader and premixed with 2.50 g Walocel for 5 min. Afterwards 31.25 g Ludox AS 40 was mixed in and kneaded for 10 min. At least 120.00 g distillated water was put into the mixture and mixed for 35 min. Then the mixture was extruded (2.0 mm). The formed extrudates were dried for 4 h at 120 °C (heating rate 3 °C/min) and calcined under air for 5 h at 500 °C (heating rate 2 °C/min).

B-MWW (1.8 wt.-% B) powder was produced by submitting 120.00 g B-MWW (1.3 wt.-% B) powder and adding a solution of premixed 3.48 g H₃BO₃ in 210 ml distilled water. This mixture was homogenized for 10 min, dried at 120 °C for 5 h and then calcinated under air at 500 °C for 5 h (heating rate 2 °C/min).

100.00 g of the obtained B-MWW powder was respectively put into a kneader and premixed with 5 g Walocel for 5 min. Afterwards 62.50 g Ludox AS 40 was mixed in and kneaded for 10 min. At least 150.00 g distillated water was put into the mixture and mixed for 50 min. Then the mixture was extruded (2.0 mm). The formed extrudates were dried for 4 h at 120 °C (heating rate 3 °C/min) and calcined under air for 5 h at 500 °C (heating rate 2 °C/min).

### Reference Example 3: Preparation of a Deboronated MWW Catalyst

A deboronated MWW (0.06 wt.-% B) powder was produced following example 1.2 of WO 2014/122152 A.

70.00 g of the obtained deboronated MWW powder was put into a kneader and premixed with 3.50 g Walocel for 5 min. Afterwards 43.75 g Ludox AS 40 is mixed in and kneaded for 10 min. At least 115.00 g distillated water was put into the mixture and mixed for 35 min. Then the mixture was extruded (2.0 mm). The formed extrudates were dried for 4 h at 120 °C (heating rate 3 °C/min) and calcined under air for 5 h at 500 °C (heating rate 2 °C/min).

### Reference Example 4: Preparation of a Ti-MWW Catalyst

Using the deboronated zeolite material having an MWW framework structure as obtained according to Reference Example 3, a titanium zeolite material having an MWW framework structure was prepared (following example 1.3 and 1.4 of WO 2014/122152 A).

The obtained titanium containing MWW (1.9 wt.-% Ti) powder (100.00 g) was put into a kneader and premixed with 5.00 g Walocel for 5 min. Afterwards 62.50 g Ludox AS 40 is mixed in and kneaded for 10 min. At least 160.00 g distillated water was put into the mixture and mixed for 50 min. Then the mixture was extruded (2.0 mm). The formed extrudates were dried for 4 h at 120 °C (heating rate 3 °C/min) and calcinated under air for 5 h at 500 °C (heating rate 2 °C/min).

### Reference Example 5: Preparation of a Sn-MWW Catalyst

Tin containing MWW (2.1 wt.-% Sn) zeolite was produced milling 100 g deboronated MWW powder and 4.07 g Sn(OAc)₂ in a Microton lab mill on intermediate level for 10 min. The obtained product was calcinated under air at 500 °C for 3 h (heating rate 2 °C/min).

80.00 g of the obtained tin containing MWW (2.1 wt.-% Sn) powder was put into a kneader and premixed with 4.00 g Walocel for 5 min. Afterwards 50.00 g Ludox AS 40 is mixed in and kneaded for 10 min. At least 160.00 g distillated water was put into the mixture and mixed for 50 min. Then the mixture was extruded (2.0 mm). The formed extrudates were dried for 4 h at 120 °C (heating rate 3 °C/min) and calcinated under air for 5 h at 500 °C (heating rate 2 °C/min).

### Reference Example 6: Preparation of a P/B-MWW Catalyst

Boron and phosphorous doped MWW zeolite was produced by submitting 120.00 g BMWW (1.4 wt.-% B) powder and adding a solution of premixed 2.24 g NH₄H₂PO₄ (ammonium dihydrogen phosphate) in 210 ml distilled water. This mixture was homogenized for 10 min, dried at 120 °C for 5 h and then calcinated under air at 500 °C for 5 h (heating rate (2 °C/min).

100.00 g of the obtained P/B-MWW (1.4 wt.-% B, 0.58 wt.-% P) powder was put into a kneader and premixed with 5.00 g Walocel for 5 min. Afterwards 62.50 g Ludox AS 40 is mixed in and kneaded for 10 min. At least 160.00 mL distillated water was put into the mixture and mixed for 50 min. Then the mixture was extruded (2.0 mm). The formed extrudates were dried for 4 h at 120 °C (heating rate 3 °C/min) and calcinated under air for 5 h at 500 °C (heating rate 2 °C/min).

### Reference Example 7: Preparation of MWW Catalysts with Different Binders

Preparation of B-MWW catalyst with ZrO₂/SiO₂ binder.

100.00 g of the obtained B-MWW (1.4 wt.-% B) powder was respectively put into a kneader and premixed with 5 g Walocel and 14.50 g ZrOH(OAc)₃ for 5 min. Afterwards 46.90 g Ludox AS 40 was mixed in and kneaded for 10 min. At least 195.00 g distillated water was put into the mixture and mixed for 35 min. Then the mixture was extruded (2.0 mm). The formed extrudates were dried for 4 h at 120 °C (heating rate 3 °C/min) and calcined under air for 5 h at 500 °C. (heating rate 2 °C/min).

Preparation of B-MWW catalyst with Al₂O₃/SiO₂ binder.

100.00 g of the obtained B-MWW (1.4 wt.-% B) powder was respectively put into a kneader and premixed with 5 g Walocel and 17.33 g Disperal P2 for 5 min. Afterwards 31.25 g Ludox AS 40 was mixed in and kneaded for 10 min. At least 195.00 g distillated water was put into the mixture and mixed for 35 min. Then the mixture was extruded (2.0 mm). The formed extrudates were dried for 4 h at 120 °C (heating rate 3 °C/min) and calcined under air for 5 h at 500 °C (heating rate 2 °C/min).

Preparation of B-MWW catalyst with Al₂O₃ binder.

100.00 g of the obtained B-MWW (1.4 wt.-% B) powder was respectively put into a kneader and premixed with 5 g Walocel and 34.67 g Disperal P2 for 5 min. Afterwards at least 205.00 g distillated water was mixed in and kneaded for 45 min. Then the mixture was extruded (2.0 mm). The formed extrudates were dried for 4 h at 120 °C (heating rate 3 °C/min) and calcined under air for 5 h at 500 °C (heating rate 2 °C/min).

### Comparative Example 1: Preparation of B-Beta Catalyst with SiO₂ Binder

100.00 g of the B-Beta (1.5 wt.-% B) powder was respectively put into a kneader and premixed with 5 g Walocel for 5 min. Afterwards 62.50 g Ludox AS 40 was mixed in and kneaded for 10 min. At least 30.00 g distillated water was put into the mixture and mixed for 35 min. Then the mixture was extruded (2.0 mm). The formed extrudates were dried for 4 h at 120 °C (heating rate 3 °C/min) and calcined under air for 5 h at 500 °C (heating rate 2 °C/min).

### Comparative Example 2: Preparation of HY-Zeolite Catalyst

2000 g of the GE 1967 HY-zeolite powder was respectively put into a kneader and mixed with 333 . . g Pural SB, 2% formic acid and 510 ml demineralized water for 30 min. Then the mixture was extruded (2.0 mm). The formed extrudates were dried for 16 h at 110 °C and calcined under air for 16 h at 500 °C.

### Comparative Example 3: Preparation of H—Al-Beta Catalyst

480 g of the GE1743d Beta-zeolite powder was respectively put into a kneader and mixed with 120 g Pural SB, 2% formic acid and 710 g of demineralized water for 45 min. Then the mixture was extruded (1.5 mm). The formed extrudates were dried for 16 h at 110 °C and calcined under air for 16 h at480 °C.

### Example 1: Catalytic Testing

The start-up phase (first 24 h) was discarded and only the following 24 h were considered in the results shown. The tests were performed in the presence of steam with a ratio of 1,1-di(p-isobutylphenyl) ethane/H₂O of 1/1.1. The results for the catalytic testing are shown in table 1.

TABLE 1
Results from catalytic testing.

| | | | Temperature | NH₃-TPD | Conversion | Selectivity |
|---|---|---|---|---|---|---|
| Example | Zeolite | Binder (20 wt.-%) | [°C] | [mmol/g] | rate [%] | PBS [%] |
| 1.1 | B-MWW | SiO₂ | 380 | 0.190 | 79 | 83 |
| 1.2 | (1.4 wt.-% B) | SiO₂ | 380 | 0.236 | 82^{a} | 73^{a} |
| 1.3 | B-MWW | SiO₂ | 380 | 0.291 | 74 | 80 |
| | (1.8 wt.-% B) | | | | | |
| 1.4 | deB-MWW | SiO₂ | 380 | 0.062 | 35 | 72 |
| 1.5 | (0.06 wt.-% B) | SiO₂ | 440 | 0.062 | 62 | 84 |
| 1.6 | P/B-MWW | SiO₂ | 380 | 0.285 | 72 | 72 |
| | (1.4 wt.-% B, | | | | | |
| | 0.58 wt.-% P) | | | | | |
| 1.7 | B-MWW | 5 wt.-% ZrO₂, | 380 | 0.330 | 63 | 42 |
| | (1.4 wt.-% B) | 15 wt.-% SiO₂ | | | | |
| 1.8 | | 10 wt.-% Al₂O₃, | 380 | 0.418 | 100 | 1 |
| | | 10 wt.-% SiO₂ | | | | |
| 1.9 | | Al₂O₃ | 380 | 0.500 | 100 | 1 |
| 1.10 | Ti-MWW | SiO₂ | 380 | 0.117 | 19 | 54 |
| 1.11 | (1.9 wt.-% Ti) | SiO₂ | 420 | 0.117 | 29 | 62 |
| 1.12 | Sn-MWW | SiO₂ | 380 | 0.114 | 53 | 59 |
| | (2.1 wt.-% Sn) | | | | | |
| Comp 1 | B-Beta | SiO₂ | 420 | 0.117 | 29 | 62 |
| | (1.5 wt.-% B) | | | | | |
| Comp 2 | HY-zeolite | SiO₂ | 350 | NA | 96 | 15 |
| Comp 3 | H-Al-Beta | SiO₂ | 180 | NA | 97 | 0.7 |

^{a}Reaction time 287.5 h

As can be gathered from the results in table 1, the best performance was achieved in example 1.1 at over 6 h at steady state with a [B—Si—O]-MWW zeolite catalyst having a boron content of 1.4 wt.-%. Example 1.2 showed 72% 1,1-di(p-isobutylphenyl) ethane conversion and 83% selectivity to isobutylstyrene. A long term performance test (example 1.2) for 287.5 h with the same catalyst sample in steady state resulted in 82% conversion and 73% selectivity to isobutylstyrene.

To investigate the influence of different acidities of the extruded catalyst samples on the conversion and isobutylstyrene selectivity, the boron content of the samples was varied from 0.06 to 1.8 wt %. An increase of boron content to 1.8 wt.-% (example 1.3) showed slightly lower conversion of 74% and 80% selectivity to isobutylstyrene. Deboronated samples in example 1.4 had a lower conversion of 35%, while still maintaining 72% selectivity to isobutylstyrene at 380 °C. An increase of conversion to 62% could be achieved through an increase in reaction temperature in example 1.5 to 440 °C. The addition of 0.58 wt.-% P in example 1.6 did further increase the acidity of the catalyst, but did not result in a better conversion or selectivity.

Another method to change the acidity of the extruded catalysts was to change the binder used in the shaping step in examples 1.7 to 1.9. The substitution of SiO₂ binder by ZrO₂ or Al₂O₃ increased the total acidity of extrudates significantly from 0.190 to 0.500 mmol/g. In this case an increased acidity did not improve the performance of the catalysts. Rather a drastic reduction of the selectivity to isobutylstyrene to 1% for examples 1.8 and 1.9 was observed.

Catalysts with Lewis centres as Sn and Ti were also tested in examples 1.10 to 1.12. Both demonstrated lower conversions and moderate selectivity to isobutylstyrene. However, an increase in reaction temperature from 380 to 420 °C in example 1.11 improved the [Ti—Si—O]-MWW performance to 29% 1,1-di(p-isobutylphenyl) ethane conversion and 62% selectivity to isobutylstyrene.

A B-Beta catalyst was investigated as a comparative example and showed an extremely low selectivity comparable to the selectivities observed when employing a binder containing Al₂O₃. Further, a HY-zeolite catalyst and an H—Al-Beta zeolite catalyst were investigated as further comparative examples. As can be seen in Table 1, both catalyst show high conversion with a low selectivity for PBS. The ratio of PBS to the by-product p-isobutylethylbenzene is 0.32 and 0.008, respectively, for Comparative Example 2 and 3. In contrast thereto, the ratio of PBS to the by-product p-isobutylethylbenzene of Example 1.1 is 11.4.

### Example 2: Regeneration of the Zeolite Catalyst

To test the regeneration of zeolite catalyst, fresh and spent catalyst were characterized regarding their boron, silicon and carbon content. Afterwards the spent sample was calcined for 5 h at 500 °C and measured again.

TABLE 2
Results for the regeneration of the catalyst.

| | | | | | | | Sel |
|---|---|---|---|---|---|---|---|
| | B, | Si, | C, | Hg-Porosity | NH3-TPD, | Conv, | IBS, |
| Catalyst | [wt.-%] | [wt.-%] | [wt.-%] | V, [ml/g] | mmol/g | [%] | [%] |
| Fresh | 1.1 | 42 | — | 1.49 | 0.190 | 79 | 83 |
| B-MWW/SiO₂ | | | | | | | |
| Spent, dried^{a} | 0.81 | 22.3 | 13.6 | 0.89 | — | — | — |
| B-MWW/SiO₂ | | | | | | | |
| Spent, calcined | 0.97 | 41 | 0.02 | 1.15 | 0.181 | 80 | 79 |
| B-MWW/SiO₂ | | | | | | | |

^{a}catalyst after 2 weeks of testing.

The regeneration of [B—Si—O]-MWW was tested in experiment 2. 10% of boron was leached during the testing and the acidity of the catalyst was also reduced by 5%. Nevertheless, the performance of regenerated catalyst was not significantly affected as can be seen in table 2.

### CITED PRIOR ART DOCUMENTS

U.S. Pat. No. 4,694,100
EP 0300498 A
EP 0316014 A
EP 0015758 A1
WO 2017/009458 A1
US 2018/134570 A1

1. A process for the production of an alkylstyrene comprising
(1) providing a feed comprising a 1,1-diarylethane compound, wherein the aryl-group is an alkylphenyl group;
(2) providing a catalyst comprising a zeolite having an MWW-type framework structure;
(3) contacting the feed provided in (1) with the catalyst provided in (2) for a catalytic cracking of the 1,1-diarylethane to the alkylstyrene and an alkylbenzene;
(4) separating the alkylstyrene from the reaction product obtained in (3).

2. A process for the production of an alkylphenyl propionic acid comprising
(1) providing a feed comprising a 1,1-diarylethane compound, wherein the aryl-group is an alkylphenyl group;
(2) providing a catalyst comprising a zeolite having an MWW-type framework structure;
(3) contacting the feed provided in (1) with the catalyst provided in (2) for a catalytic cracking of the 1,1-diarylethane to the alkylstyrene and an alkylbenzene;
(4) optionally separating the alkylstyrene from a reaction product obtained in (3);
(5) preparing a mixture containing CO, the alkylstyrene-containing reaction product obtained in (3) or the alkylstyrene obtained in (4), and optionally a solvent system;
(6) providing a carbonylation catalyst;
(7) contacting the mixture obtained in (5) with the carbonylation catalyst provided in (6) for converting the alkylstyrene to an alkylphenypropionic acid.

3. The process according to claim 1, wherein in (1) the alkylphenyl group is selected from the group consisting of o-isobutylphenyl, misobutylphenyl, and p-isobutylphenyl.

4. The process according to claim 1, wherein the zeolite in (2) is selected from the group consisting of PSH-3, SSZ-25, ERB-1, MCM-22, ITQ-1, SSZ-25, [Ga—Si—O]-MWW, [B—Si—O]-MWW, [Sn—Si—O]-MWW, [AI-Si—O]-MWW, [Zr—Si—O]-MWW, [V—Si—O]-MWW, [Nb—Si—O]-MWW, [Ta—Si—O]-MWW, [Cr—Si—O]-MWW, [Mo—Si—O]-MWW, [W—Si—O]-MWW, [Mn—Si—O]-MWW, [Fe—Si—O]-MWW, [Co—Si—O]-MWW, [NiSi—O]-MWW, [Zn—Si—O]-MWW, [Ga—Si—O]-MWW, [Ge—Si—O]-MWW, [In—Si—O]-MWW, [Pb—Si—O]-MWW, [Cu—Si—O]-MWW, [Ti—Si—O]-MWW, and mixtures of two or more thereof.

5. The process according to claim 1, wherein the zeolite in (2) comprises a [B—Si—O]-MWW zeolite, wherein the [B—Si—O]-MWW zeolite has a boron content in an range of from 0.1 to 5 wt.-% B calculated as the element and based on 100 wt.-% of B, Si, and O contained in the zeolite.

6. The process according to claim 1, wherein the zeolite in (2) comprises a [B—Si—O]-MWW zeolite, wherein the [B—Si—O]-MWW zeolite has been deboronated.

7. The process according to claim 6, wherein prior to the deboronation, the [B—Si—O]-MWW zeolite initially contained from 0.1 to 5 wt.-% B calculated as the element and based on 100 wt.-% of B, Si, and O contained in the zeolite.

8. The process according to claim 1, wherein in (2) the catalyst is provided as a shaped body.

9. The process according to claim 8, wherein the shaped body contains a binder.

10. The process according to claim 9, wherein the binder is substantially free of Al₂O₃.

11. The process according to claim 1, wherein the process includes a step of regenerating the catalyst provided in (2), and contacted with the feed in (3), at regular intervals, wherein the catalyst is regenerated by calcination at a temperature in a range of from 300 to 850 °C.

12. The process according to claim 1, wherein in (2) the zeolite having the MWW-type framework structure is prepared by a process comprising
(a) hydrothermally synthesizing an MWW precursor from a synthesis mixture containing at least one silicon source, at least one boron source, and at least one template compound, to obtain the MWW precursor in its mother liquor;
(b) separating the MWW precursor from its mother liquor;
(c optionally drying the MWW precursor separated according to (b);
(d) calcining the MWW precursor obtained from (b) or (c), and obtaining a [B—Si—O]-MWW.

13. The process according to claim 12, wherein in (2) the zeolite having the MWW framework type is prepared by a process further comprising
(i) preparing a [B—Si—O]-MWW according to the process comprising steps (a) to (d);
(ii) deboronating the [B—Si—O]-MWW by treating the [B—Si—O]-MWW with a liquid solvent system thereby obtaining a deboronated zeolite having the MWW-type framework structure;
wherein the liquid solvent system is selected from the group consisting of water, monohydric alcohols, polyhydric alcohols, and mixtures of two or more thereof, and wherein said liquid solvent system does not contain an inorganic or organic acid or a salt thereof, the acid being selected from the group consisting of hydrochloric acid, sulfuric acid, nitric acid, phosphoric acid, formic acid, acetic acid, propionic acid, oxalic acid, and tartaric acid.

14. The process according to claim 13 further comprising
(iii) post-treating the deboronated zeolite having the MWW-type framework structure obtained from (ii) by a process comprising
(iii.1) separating the deboronated zeolite having the MWW-type framework structure from the liquid solvent system;
(iii.2) optionally drying the separated deboronated zeolite having the MWW-type framework structure;
(iii.3) optionally calcining the deboronated zeolite having the MWW-type framework structure obtained from (iii.1) or (iii.2).

15. The process according to claim 13 further comprising
(iv) incorporating at least one heteroatom into the deboronated zeolite having the MWW-type framework structure thereby obtaining a zeolite having the MWW-type structure containing at least one heteroatom by a process comprising
(iv.1) preparing a synthesis mixture containing the deboronated zeolite having the MWW-type framework structure obtained according to (ii) or (iii), a template compound, and at least one source of at least one heteroatom;
(iv.2) hydrothermally synthesizing the zeolite having the MWW-type structure containing at least one heteroatom from the synthesis mixture obtained from (iv.1) thereby obtaining the zeolite having the MWW-type structure containing at least one heteroatom in its mother liquor;
(iv.3) separating the zeolite having the MWW-type structure containing at least one heteroatom from its mother liquor;
(iv.4) optionally drying the zeolite having the MWW-type structure containing at least one heteroatom separated according to (iv.3);
(iv.5) optionally calcining the zeolite having the MWW-type structure containing at least one heteroatom obtained from (iv.3) or (iv.4).
